(19) United States
(12) Patent Application Publication
(10) Pub. No.: US 2007/0154538 A1
(43) Pub. Date: Jul. 5, 2007

(54) Removal of fat cells by PDT

(75) Inventors: Wolfgang Neuberger, Labuan (MY); Volker Albrecht, Jena (DE)

Correspondence Address: BOLESH J. SKUTNIK;CERAMOPTEC INDUSTRIES, INC., 515 SHAKER RD., EAST LONGMEADOW, MA, 01028, US

(73) Assignees: CeramOptec GmbH; CeramOptec Industries Inc.

(21) Appl. No.: 11/321,105

(22) Filed: Dec. 29, 2005

(51) Int. Cl. A61K 31/409; A61K 31/555; A61K 9/127

(52) U.S. Cl. 424/450; 514/185; 514/410

(57) ABSTRACT

A non-surgical, minimally invasive PhotoDynamic Therapy (PDT) method for destruction of undesirable fat cells in the body, which cause obesity, obesity related health problems, and undesirable body contours. In this method photosensitizing agents are formulated into a liposomal formulation or into other delivery means for administering the drug systemically, directly by injection and/or topical application. The photosensitizing formulation is selectively targeted at adipose tissue in the subcutaneous layer. Photosensitized adipose cells are illuminated with a predetermined wavelength of light energy known to initiate a photo cytotoxic effect. The light energy for photoactivation of the photosensitizing agent may be applied interstitially through optical fibers with or without a diffuser tip. Alternatively, a photosensitizing agent is selected to have an activating wavelength which permits external irradiation through the skin. The method ensures safe and permanent fat reduction, minimizes trauma to the area of treatment and also hastens the healing process. A lasting and permanent effect is provided in regions of fat accumulation as the adipose cells are permanently destroyed. This approach is also effective for fat deposits which do not respond to other treatments.

### BACKGROUND OF THE INVENTION

1. Field of the Invention

This invention relates to the field of (photodynamic therapy) PDT and cosmetic surgery, and, in particular, relates to the application of PDT methods for the treatment of obesity for aesthetic reasons and also to prevent medical complication.

2. Invention Disclosure Statement

Adipose tissues are specialized loose connective tissues that function as major storage sites for fat in the form of triglycerides. In mammals adipose tissues are of two types, namely, white adipose tissue (WAT) and brown adipose tissue (BAT). Adipose tissues have different functions, white adipose tissue serves three functions: namely, heat insulation, mechanical cushion, and most importantly, as source of energy. While brown adipose tissues (BAT) are found in hibernating and neonates for regulating body temperature, BAT is highly vascularized and hence darkly colored.

Obesity and body overweight results from excessive fat accumulation in the adipocytes especially in the white adipose tissue. Several factors contribute to the development of obesity which includes genetics, environment, physiology, psychology, energy-rich food, limited excises and sedentary lifestyle, etc.

Research in animal models has shown that genetic anomalies as one major cause for obesity. ‘ob’, ‘db’ and ‘Ay’ are a few genes reported to be associated with obesity. Leptin is a hormone normally produced by “ob” gene in fat cells which stimulates lipolysis, thus mobilizing fat deposits. Insufficient/insensitivity to leptin can also cause obesity. This can be because of defective “ob” gene, which, cannot synthesis functional leptin in fat cells or it can be due to lack of leptin receptor on hypothalamus.

Hormones like Insulin also play an important role in fat deposition. Insulin is found to activate lipogenesis (fat anabolism), while inhibiting lipolysis (fat catabolism) and is also involved in fat cell differentiation. Therefore it is also called the fattening hormone.

Fat is stored in the body at two places: subcutaneously and viscerally, visceral fat which is deposited around the internal organ is easy to lose compared to the subcutaneous fat. Fat accumulation in an individual varies depending upon his/her genetic makeup and hormones, basically two types are reported based on the gender: Android pattern mostly associated with men, where the fat is deposited over midsection, abs, obliques and lower back; while fat deposition on lower body, on hips, thighs, tummy and below the navel is associated with women and is termed as gynoid pattern. Both of these patterns can be changed based on age and hormone fluctuation in both the genders.

In humans, obesity results in many health problems like hypertension, type 2 diabetics, cardiovascular disease, joint pains, sleep apnea and many more. In addition to the medical consequences, obesity has serious social effects. Today younger and older generations are becoming more conscious about their physical appearance. The desire of individuals to lose weight to achieve a slim figure has become an obsession, and, billions of dollars are spent on weight loss products and programs each year.

To control fat deposition and weight gain it is important to control the processes of fat synthesis and its accumulation in adipose cells. In prior art different treatment methods have been developed to control obesity.

Treatment for fat reduction/ obesity includes:

Regular exercise for building body muscle tissue while losing fat; controlling daily caloric intake by using low fat, high complex carbohydrate and high fiber content diets. This method of weight loss is slow, progressive and time consuming.

Diet drugs, for example, Orlistat, Sibutramine, amphetamines, etc., are used in controlling excess food intake. Most of these drugs lose their effectiveness over time, thus requiring increased dosage and they can be addictive and dangerous. Mostly these drugs are non-selective in their action.

Other methods include surgical methods, liposuction, laser therapy, many of which are invasive methods used for treating obesity.

Weight loss and obesity control by dieting and diet drugs have a temporary effect, lasting only during the treatment period. Studies have shown that in most cases such a patient regains more weight after he/she stops dieting or taking diet drugs. While other methods like surgery, liposuction, laser therapy, etc. are all more invasive methods that may lead to excessive bleeding, infection, hospitalization, and require regular follow up after surgery, etc.

Liposuction/lipoplasty is a popular cosmetic surgery which is useful in men and women who have normal weight but involves isolated pockets of fat in certain parts of the body giving a disproportionate effect. To undergo liposuction the skin elasticity must be in good shape to get a smooth contour after surgery; otherwise, additional skin tightening surgery will need to be done. Moreover, it is not a substitute for overall weight loss. Other side effects are bleeding, infection, contour irregularity including depression or wrinkling. Patient needs to wear specially designed garment like elasticized bandage for certain period of time after liposuction.

A number of radiation techniques have been proposed as less invasive methods. For example, ultrasonic probes, radio frequency devices have also been used for fat cell elimination. These methods tend to be invasive, not selective and require incisions in the skin which can lead to scarring, healing and infection problems.

In U.S. Pat. No. 6,354,297 by Eiseman combinations of apoptosis inducing factors like ultraviolet light and chemical substances which induce apoptosis in fat cell directly are described. In this invention a device is implanted temporarily adjacent to fatty tissue which delivers the chemical or light signals to fat cells for inducing apoptosis.

Laser surgery is another method to control obesity; for example, U.S. Pat. No. 5,644,585 by Mitchell et al. discloses uses of laser for tissue ablation and emulsifying biological material. Mitchell et al. have developed a Nd: YAG medical laser systems which can be used for removal of fatty tissue. Another example is U.S. Pat. No. 6,605,080 by Altshuler et al which discloses methods for selective removal of lipid rich tissue using light source like laser, diode laser, tungsten lamp, etc. with wavelengths in the range 880 nm to 935 nm; 1150 nm to 1230 nm; 1690 nm to 1780 nm; and 2250 nm to 2450 nm. Wavelength of lower band range; 900 nm to 930 nm and 1210 nm to 1230 nm band is most preferred for fat tissue ablation. Laser light is applied externally and liquidized lipids are removed by use of suction. A major point is to employ a cooling system to avoid heat damage to skin and nearby cells. Energy levels to emulsify or ablate fats can be quite high raising local temperatures as noted in some the earlier references, so that thermal damage to tissue nearby can be a problem. Further these methods of using different energy sources for destruction of fat cells are very crude and nonspecific causing trauma to the surrounding cells, initiating inflammatory action and scarring to some extent.

US Patent Application No. 20050085455 by James C. Chen describes the use of transcutaneous Photodynamic therapy for adipocyte reduction. In this method photosensitizing agents are targeted at fat cells for controlling adiposity. A main drawback is low fluence of light and long hours of irradiation required to reduce concerns for thermal and photochemical damage to nearby tissue and skin.

The disadvantage in these current approaches are, each is invasive, can involve excessive bleeding and, possibly more significantly, each is nonselective, leading to removal of non targeted cells in the subcutaneous layer, damaging blood capillaries, causing burns, and other physical traumas to the near by cells.

Thus, there remains a need for improved treatment methods with selective elimination of fat deposition to control obesity and also to maintain a slim physique. In the present invention a minimally invasive PDT treatment is disclosed for losing excess fat in humans without surgery or scarring and requiring less recovery time. This invention is also useful for isolated fat removal in thighs, eyelids, belly, buttock, cheek, etc. for cosmetic purposes as well as for medical benefit.

### OBJECTIVES AND BRIEF SUMMARY OF THE INVENTION

It is the objective of this invention to provide a suitable Photodynamic Therapy method for effectively treating obesity in organisms by fat cell destruction.

It is also an objective of this invention to provide a method of PDT for contour correction, for improving cosmetic appearance by reducing fat accumulation around hips, buttocks, stomach, thighs, arm, back and hamstrings and also to remove excess fat of the face (under the eyes), neck, chin and jowls.

It is another objective of this invention to use photosensitizing agents or precursors to photosensitizers with a photosensitizing agent delivery system for topical and systemic application.

It is another aim of the present invention to provide a PDT method to directly affect the sub-dermal fatty tissue.

It is further objective of this invention to enhance or increase the lipolysis by increasing metabolic rate.

It is still further objective of this invention to provide a method of using laser or non-laser light source for photoactivation of photosensitizer accumulated in targeted adioposities.

It is another aim of the present invention to provide an optical fiber with a diffuser tip for delivering the light source to the targeted fat cells.

It is also another aim of the present invention to provide a method of PDT that directly acts on the fatty tissue in the body, causing emulsification and then elimination by absorption and removal by lymphatic system.

Briefly stated, the present invention provides a non-surgical, minimally invasive PhotoDynamic Therapy (PDT) method for destruction of undesirable fat cells in the body, which cause obesity, obesity related health problems, and undesirable body contours. In this method photosensitizing agents are formulated into a liposomal formulation or into other delivery means for administering the drug systemically, directly by injection and/or topical application. The photosensitizing formulation is selectively targeted at adipose tissue in the subcutaneous layer; photosensitized adipose cells are illuminated with a predetermined wavelength of light energy known to initiate a photo cytotoxic effect. The light energy for photoactivation of the photosensitizing agent may be applied interstitially through optical fibers with or without a diffuser tip. Alternatively, a photosensitizing agent is selected to have an activating wavelength which permits external irradiation through the skin. The method ensures safe and permanent fat reduction, minimizes trauma to the area of treatment and also hastens the healing process. A lasting and permanent effect is provided in regions of fat accumulation as the adipose cells are permanently destroyed. This approach is also effective for fat deposits which do not respond to other treatments.

The above and other objects, features and advantages of the present invention will become apparent from the following description read in conjunction with the accompanying drawings.

### DETAILED DESCRIPTION OF PREFERRED EMBODIMENTS

A fat cell is also called an adipocyte which is a specialized loose connective tissue that when found in the hypodermis is referred to as subcutaneous fat., Fat cells deposited on internal organs are termed as visceral fats. Adipocyte sizes can vary from 20-200 μm in diameter, and are capable of expanding 20-fold in diameter and by several thousand fold in volume, by accumulating more triglycerides. Adipose tissue expansion may occur by adipocyte hypertrophy and/or hyperplasia. Fat cells reaching a critical size can trigger events that result in proliferation of adipocytes.

A recent World Health Organization report shows a staggering one billion of world's population of 6.5 billion is over weight, if the current trend continues by 2015 there will be 1.5 billion over weight people in the world.

Obesity is the term used to define the condition which results from excessive fat deposition in the adipocyte. In an obese or overweight individual, lipogenesis is more active than lipolyis. In other words energy consumption is more prevalent than energy spent. The imbalance in lipid metabolism pathways can be due to many factors: which can include genetic factors, environmental factors, lifestyle, eating behavior, and an increasing sedentary life style.

Overweight and obesity is a major health concern. Due to technology development, man's life has become luxurious and sedentary, involving less physical activity leading to less energy spent than consumed. Health problem is not the only risk faced by overweight individuals; they are also subjected to social and emotional problems.

Individuals desire to lose weight not just for reducing the risk of getting afflicted by obesity associated diseases, but, also for improving one's cosmetic appearance. Everyone today is more concerned about their looks, the desire to lose weight to achieve perfect slim figure has become an obsession, and, billions of dollars are spent on weight loss products and programs each year.

In order to inhibit/control fat accumulation in the body it is important to either act directly on the adipocytes or can target the different cell components along the fat metabolic pathway. The overall aim of this invention is to rid the over weight individual of undesired body fat cells without harmful reactions experienced by present fat-removal techniques and procedures. The present invention can be used for cosmetic surgery and to improve deteriorating health condition of an obese individual suffering from obesity related disease. In particular, it is a minimally invasive procedure which can safely be used to treat HIV and Aids patients who can benefit medically and psychologically from undesired and unhealthy accumulation of fat cells as a consequence of their ailment.

In the present invention, photodynamic therapy is used for fat cell reduction. PhotoDynamic Therapy (PDT) is a new treatment modality used for treating certain types of cancers and other hyperproliferative tissue diseases.

PDT is found to be effective in controlling cancerous growth with minimal side effects. In this method, first step is to administer photosensitizing agents either topically or systemically followed by illumination of the targeted region with a suitable light source whose wavelength matches the absorption spectrum of the photosensitizing agent. The photoactivated photosensitizer produces highly cytotoxic, oxygen-derived species most notably, singlet oxygen to destroy targeted fat cells. A photosensitizing agent can be a photosensitizer, a precursor to a photo sensitizer or a derivative of a photosensitizer. Among the preferred photosensitizers are dihydro- and tetra hydro- tetrapyrroles, which are examples of hydrophobic macrocycle porphyrin species that are favored for reduction of fat cell applications.

In this invention, the method includes the step of administering to the subject a therapeutically effective amount of a photosensitizing agent containing a photosensitizer or a precursor to a photosensitizer, having a characteristic light absorption waveband. Alternatively, a photosensitizing agent delivery system can be used which includes but is not limited to liposome, pegylation, microsphere and or nanoparticles to aid in delivering the photosensitizing agent, having a characteristic light absorption waveband. This is administered to the patient locally or systemically. The photosensitizer may be targeted at adipose tissue in order to avoid accumulation and photosensitivity in other non-targeted healthy cells.

In order to provide this treatment, the fatty tissue area is identified as compared with cellulite tissue since the treatments of these are different. Because of less vascularized tissue in the fatty area, the photosensitizer will remain there longer Identification of the treatment area may use direct observation, physical examination, sonography, and/or thermosensors, for example.

The present invention also relates to photosensitizing agents comprising a pharmaceutically acceptable carrier system. A suitably designed carrier system is used to deliver photosensitizer to fat cells located in subcutaneous layer of the skin. It also protects the photosensitizing agent from immune system recognition and enzymatic degradation, prior to reaching targeted cells.

In one embodiment of this invention, a liposome is used to deliver the photosensitizing agent, a hydrophobic photo sensitizer (PS), or a PS precursor or a PS derivative, to the sub-dermal fat cell. Liposomal formulations enhance absorption of photosensitizer though epidermis when applied topically because of their hydrophilic and lipophilic characteristics which help them pass through skin surface easily. This ensures that required concentration of photosensitizer is accumulated in target cell which in turn gives higher quantum yield when photoactivated.

In a preferred embodiment the hydrophobic, photosensitizer temoporfin (mTHPC) can be used in two different formulations for being administered to fat cells either systemically or topically. mTHPC is formulated either in a liposomal formulation or in an aqueous-organic mixture.

A hydrophobic photosensitizer such as mTHPC used in the present invention is found to show low solubility level in the triglycerides the main component of adipose cells, thus a high concentration of temoporfin is accumulated in the active part of the adipose cell, thus facilitating specific destruction of fat cells in the body when compared to other photosensitizers used for fat cell destruction y PDT.

In one of the embodiments a liposomal formulated mTHPC is administered to an obese patient, the photosensitizer is targeted at adipose cells. The liposomal formulation of hydrophobic photosensitizer mTHPC facilitates easy transport into adipose cells. The cells are then irradiated with a wavelength around 652 nm. In another embodiment, mTHPC can be formulated in an aqueous alcoholic solution for systemic administration.

Before administering PDT, the area of treatment is marked to evaluate excess adipose tissues in the body that needs to be removed. Once this is determined, photosensitizer is administered either locally or topically, followed by illumination of the marked region using a light source with a wavelength matching the absorption spectrum of chosen photosensitizer. The light source can be applied externally, or interstitially. Light sources used can be a laser, a LED, a lamp or any other source which is known in the field to activate the selected photosensitizer.

In yet another embodiment PDT can be utilized for cosmetic weight loss programs and to improve body contour. This invention can be effectively used to reduce fat accumulated in selected body parts like thighs, abdomen, buttock, arm, back and also to remove excess fat of eyelids, cheek, neck, chin etc for cosmetic purposes and the psychological gains that these can bring.

The present invention is further illustrated by the following examples, but is not limited thereby.

### EXAMPLE 1

Fat reduction for aesthetic purposes:

The fat accumulations in the areas of cosmetic concern are first marked before starting the treatment to exactly estimate amount of fat cells which need to be reduced/eliminated by the PDT procedure. Once the fat cells which need to be destroyed are marked, the individual is given a required dose of suitably formulated photosensitizer. In this embodiment a hydrophobic photosensitizer (mTHPC) is formulated into a liposome for topically application. The time required for maximum concentration of the photosensitizer in the fat cells is predetermined. After waiting for this time interval, the fat cells are then irradiated with a wavelength around 652 nm. An optical fiber with a diffuser tip is used for delivering irradiation light to the target site.

In another embodiment of this invention the same photosensitizer mTHPC is formulated for systemic application. An adequate time for achieving preferential concentration within the fat cells is passed, then the photosensitized fat cells are irradiated with light source of wavelength 652 nm using a optical fiber with diffisuser tips.

The irradiation light to the target area is delivered through an optical fiber with a preferred diffuser tip for uniform illumination of the region. The fiber can be inserted into the subcutaneous layer through a tiny incision in the skin to access presensitized fat cells. Alternatively, irradiation can be applied externally, using a suitable light source with a long wavelength which penetrates the different skin layers and reaches the hypodermal layer. The selected wavelength was chosen because it also photoactivates the preselected photo sensitizer.

### EXAMPLE 2

Alternative Photosensitizer:

The fatty tissues in the body which need to be removed by the PDT method is first marked to estimate approximately the amount of fat cells which need to be eliminated. A required dose of hydrophobic photosensitizer, e.g., 5, 10, 15, 20-tetrakis (m-hydroxyphenyl) bacteriochlorin (mTHPBC) is administered to the patient systemically or topically. After a minimum time gap, the marked fatty tissue areas are irradiated externally using laser energy of wavelength 740 nm. Delivery of radiation was fulfilled by means of optical fiber. The undesired fat cells are destroyed by the photoactivated photosensitizer.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A method of reducing unwanted fat cells in selective areas of an organism comprising the steps of:
identifying said selective areas to be treated;
administering at least once a photosensitizing agent according to claim 12 to said organism;
allowing a sufficient time for said photosensitizing agent to accumulate in fat cells and be eliminated in otherwise healthy tissue; and
selectively activating of said photosensitizing agent in said selective areas to reduce/eliminate fat cells.

2. The method of reducing unwanted fat cells according to claim 1 wherein said administering is by local or systemic application.

3. The method of reducing unwanted fat cells according to claim 1 wherein said selectively activating is by means of electromagnetic radiation, whose wavelength overlaps with an absorption peak of said photosensitizing agent.

4. The method of reducing unwanted fat cells according to claim 3 wherein said electromagnetic radiation is applied externally to said organism and to said selective areas.

5. The method of reducing unwanted fat cells according to claim 4 wherein said electromagnetic radiation is laser radiation.

6. The method of reducing unwanted fat cells according to claim 5 wherein said laser radiation is applied by means of at least one optical fiber.

7. The method of reducing unwanted fat cells according to claim 6 wherein said optical fiber has an output tip having a diffusing device thereon.

8. The method of reducing unwanted fat cells according to claim 1 wherein said fat cells are adipose tissue.

9. The method of reducing unwanted fat cells according to claim 8 wherein adipose tissue is white adipose tissue.

10. The method of reducing unwanted fat cells according to claim 1 wherein said fat cells are adipocytes.

11. The method of reducing unwanted fat cells according to claim 1 wherein said photosensitizing agent is able to avoid immune system detection and enzymatic degradation.

12. A photosensitizer formulation for reducing adipose tissue comprising:
a hydrophobic macrocycle porphyrin species; and
a carrier.

13. The photosensitizer formulation according to claim 12 wherein said photosensitizing agent is selected from the group consisting of dihydro tetrapyrroles and tetrahydro tetrapyrroles.

14. The photosensitizer formulation according to claim 13 wherein said carrier is a liposomal carrier.

15. The photosensitizer formulation according to claim 13 wherein said photosensitizing agent is temoporfin.
